SEQUENCE LISTING (13 sequences)

SEQ ID NO: 1 - DNA, 1802 bp, Gallus gallus
  chicken soluble epoxide hydrolase (sEH)
ggaggtcaccggttcgagaccgggcggtggcggaggtcatggcgcggaggtttgcgttgttcgatttaggcggagtgctcttcgggcccggcctgcagcactttctgggctcctgcgagcggagctacgccttgcccaggaatttcttgcgggatgtcctgtttgcaggtggctctgacagcccccacgccaaggtgatgcgggggcagatcaccctgtcccagctcttcttggaggtggacgagggttgcaggcagcacgcctccactgcgggcatcacgttaccaaccaccttttccatcgcccatgtgtttgaggagatggcagccaagggcaccctcaatgccccactgctgcgggctgccagcatgctccggaggaatgggtttaagacctgtgtcttcaccaacaactgggtagatgacagcatggggcggctcttcacctcctctctgctgaccgtggtgcagcggcacttcgacctgctcattgaatcctgccgtgtggggctgcacaagccggacccccgcatctacacctacgccctggaggtgctgcaggcacagccacaggaggtgatctttctggatgacattggggagaacttgaagccggcccgagagatgggcatggccaccatactcgtcagggacactgataccgtcctgaaggagctgcaggagctgtcaggcgtgcagcttctccagcaagaagaaccattgccgaccacctgtgatccagccaccatgagccatggatacgttcccatccggcctggtgtgcagctgcatttcgtggagatggggcacggccctgctatctgcctgtgccatggcttccccgagtcctggctctcctggcgctaccagattcctgccctggctgatgctggcttccgtgttattgctttggagatgaagggctatggcgagtccacagcaccgccagagatagaagaatattcccaggagcagatctgtaaggacctgaccattttcctggacaaactgggcatcccacaagccgtgttcatcggccacgactggggtggtgcagtggtctggaacatggccctcttctaccccgagagagtgagggccgtggcctcactgaacaccccataccgaccagcagaccccacagtggacatcgtggagaccatgaaaagcttccctatgtttgattaccagttctacttccaggagccaggcgttgcagaggctgagctggagaaggacattggccgtaccctgaaagcccttatccgttccacccgcccagaggaccgcctgcactcggtgcccggcctgcttggtgtccaggagcgaggggggctgctggtcggcttcccagaggacattcctgagagcctcatcctgcacggtgctgagctgcagtactacatcgagcgcttccagaggtctggcttcaggggtcctctgaattggtaccggaacatgagacccaactggcgctgggcactctcagccaaggacaggaagatcctcatgccggcgctgatggtgacagcggggaaggacgtggtgttgctccccagcatgagcaagggcatggaggagtggatcccacagctccgccgggggcacctggaggcgtgtggccattggacacagatggagaggccagcagccctgaacaggatcctggtggagtggttggaggggctccccccggatggggccatgctgagggtatcccggctgtgagcatccctgcagctccatcccagggctgccctcgccctggggttatgctgggaggggggaaaaaaaaaaaaaaaa

SEQ ID NO: 2 - protein, 562 aa, Gallus gallus
  chicken soluble epoxide hydrolase (sEH)
MARRFALFDLGGVLFGPGLQHFLGSCERSYALPRNFLRDVLFAGGSDSPHAKVMRGQITLSQLFLEVDEGCRQHASTAGITLPTTFSIAHVFEEMAAKGTLNAPLLRAASMLRRNGFKTCVFTNNWVDDSMGRLFTSSLLTVVQRHFDLLIESCRVGLHKPDPRIYTYALEVLQAQPQEVIFLDDIGENLKPAREMGMATILVRDTDTVLKELQELSGVQLLQQEEPLPTTCDPATMSHGYVPIRPGVQLHFVEMGHGPAICLCHGFPESWLSWRYQIPALADAGFRVIALEMKGYGESTAPPEIEEYSQEQICKDLTIFLDKLGIPQAVFIGHDWGGAVVWNMALFYPERVRAVASLNTPYRPADPTVDIVETMKSFPMFDYQFYFQEPGVAEAELEKDIGRTLKALIRSTRPEDRLHSVPGLLGVQERGGLLVGFPEDIPESLILHGAELQYYIERFQRSGFRGPLNWYRNMRPNWRWALSAKDRKILMPALMVTAGKDVVLLPSMSKGMEEWIPQLRRGHLEACGHWTQMERPAALNRILVEWLEGLPPDGAMLRVSRL

SEQ ID NO: 3 - protein, 555 aa, Homo sapiens
  human soluble epoxide hydrolase (sEH)
MTLRAAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGSGPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 4 - protein, 554 aa, Mus musculus
  mouse soluble epoxide hydrolase (sEH)
MALRVAAFDLDGVLALPSIAGAFRRSEEALALPRDFLLGAYQTEFPEGPTEQLMKGKITFSQWVPLMDESYRKSSKACGANLPENFSISQIFSQAMAARSINRPMLQAAIALKKKGFTTCIVTNNWLDDGDKRDSLAQMMCELSQHFDFLIESCQVGMIKPEPQIYNFLLDTLKAKPNEVVFLDDFGSNLKPARDMGMVTILVHNTASALRELEKVTGTQFPEAPLPVPCNPNDVSHGYVTVKPGIRLHFVEMGSGPALCLCHGFPESWFSWRYQIPALAQAGFRVLAIDMKGYGDSSSPPEIEEYAMELLCKEMVTFLDKLGIPQAVFIGHDWAGVMVWNMALFYPERVRAVASLNTPFMPPDPDVSPMKVIRSIPVFNYQLYFQEPGVAEAELEKNMSRTFKSFFRASDETGFIAVHKATEIGGILVNTPEDPNLSKITTEEEIEFYIQQFKKTGFRGPLNWYRNTERNWKWSCKGLGRKILVPALMVTAEKDIVLRPEMSKNMEKWIPFLKRGHIEDCGHWTQIEKPTEVNQILIKWLQTEVQNPSVTSKI

SEQ ID NO: 5 - protein, 560 aa, Xenopus tropicalis
  frog soluble epoxide hydrolase (sEH)
MAARRFVLFDLGGVLLTPGPQVAFQRLERSLSLPSGFLQNVFVRSGSEGPFARAERGQIPFSKFIAEMDKECKAFAEESGVSLPDSFSLEQTFHGMFESGGINKPMLKAAVTLRHHGFKTCVLTNNWIDDSPQRSHSAELFSSLNRHFDLVVESCRTGMRKPETQIYDYALKMLKANPKETIFLDDIGANLKPAREMGIATVLVKDTETALKELQALSGVPLFENEEVTPVPANPDNVTHGSVTVKPGVQLHYVEMGNGPVICLCHGFPESWYSWRFQIPALADAGFRVIAFDMKGYGDSSAPQEIEEYSQEQICKDLVSFLDVMGISQASFIGHDWGGAVVWNMALFYPERVRAVASLNTPFFTSDPGVNALERIKANPIFDYQLYFQEPGVAEAELEKDLERTFKVFFRGSSEKDRLATSLTTMNVRERGGILVGTDEDPPLSSIINEADLQYYVAQFKKSGFRGPLNWYRNMQRNSEWNISAHGWKILVPALMVTAGKDFVLLPIMTKGMENLIPNLSRGHIEECSHWTQMERPAAVNGILIKWLAEVHNLPVTSKL

SEQ ID NO: 6 - DNA, 20 bp, Artificial Sequence
  Description of Artificial Sequencesynthetic 5′ RACE nested primer 5R1
ctgaagccagacctctggaa

SEQ ID NO: 7 - DNA, 28 bp, Artificial Sequence
  Description of Artificial Sequencesynthetic 5′ RACE nested primer 5R2
ccgtgcaggatgaggctctcaggaatgt

SEQ ID NO: 8 - DNA, 23 bp, Artificial Sequence
  Description of Artificial Sequencesynthetic 5′ RACE nested primer 5R3
ccctcgctcctggacaccaagca

SEQ ID NO: 9 - DNA, 24 bp, Artificial Sequence
  Description of Artificial Sequencesynthetic 3′ RACE nested primer 3R1
aagcccttatccgttccacccgcc

SEQ ID NO: 10 - DNA, 23 bp, Artificial Sequence
  Description of Artificial Sequencesynthetic 3′ RACE nested primer 3R2
tgcttggtgtccaggagcgaggg

SEQ ID NO: 11 - DNA, 26 bp, Artificial Sequence
  Description of Artificial Sequencesynthetic 3′ RACE nested primer 3R3
acattcctgagagcctcatcctgcac

SEQ ID NO: 12 - DNA, 32 bp, Artificial Sequence
  Description of Artificial Sequencesynthetic PCR primer CHXF
gcggccgcatggcgcggaggtttgcgttgttc

SEQ ID NO: 13 - DNA, 33 bp, Artificial Sequence
  Description of Artificial Sequencesynthetic PCR primer CHXR
gcggccgctcacagccgggataccctcagcatg

(19) United States
(12) Patent Application Publication
(10) Pub. No.: US 2008/0188554 A1
(43) Pub. Date: Aug. 7, 2008

(54) METHODS OF TREATING AVIAN HYPERTENSION

(75) Inventors: Bruce D. Hammock, Davis, CA (US); Todd R. Harris, Sacramento, CA (US); Christophe Morisseau, West Sacramento, CA (US); Rosemary L. Walzem, College Station, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Regents of the University of California Office of Technology Transfer, Oakland, CA (US); Texas A&M University, College Station, TX (US)

(21) Appl. No.: 11/961,881

(22) Filed: Dec. 20, 2007

Related U.S. Application Data
(60) Provisional application No. 60/871,904, filed on Dec. 26, 2006.

(51) Int. Cl. A61K 31/558; C07H 21/04; C12N 9/14

(52) U.S. Cl. 514/475; 536/23.2; 435/195

(57) ABSTRACT

The present invention provides methods of treating avian pulmonary hypertension syndrome by administering to an avian subject a therapeutically effective amount of an inhibitor of soluble epoxide hydrolase (“sEHI”), alone or co-administered in combination with a cis-epoxyeicosantrienoic acid (“EET”). The invention also provides nucleic acid and amino acid sequences of an avian soluble epoxide hydrolase.

### CROSS-REFERENCE TO RELATED APPLICATIONS

The present application claims the benefit of U.S. Provisional Application No. 60/871,904, filed Dec. 26, 2006, the entire disclosure of which is hereby incorporated herein by reference.

### STATEMENT AS TO RIGHTS TO INVENTIONS MADE UNDER FEDERALLY SPONSORED RESEARCH OR DEVELOPMENT

This invention was made with government support under National Institute of Environmental Health Sciences (NIEHS) Grant R37ES002710, NIEHS Superfund Basic Research Program Grant P42 ES004699, and NIEHS Advanced Training in Environmental Toxicology Grant T32 ES007059, awarded by the National Institutes of Health. The government has certain rights in the invention.

### BACKGROUND OF THE INVENTION

Pulmonary hypertension syndrome (PHS) in chicken includes a number of conditions such as right-sided congestive heart failure, hypoxemia, pulmonary hypertension (PH) and acites (Wideman, R. F. Avian Poult. Rev. 11:21-43 (2000)). It is estimated that PHS afflicts 4% of the broilers worldwide. Interestingly, broilers are more susceptible to PHS than Leghorns (Julian, R. J. Avian Pathol. 22:419-454 (1993)). One contributing factor is thought to be impaired endothelium dependent vasodilation in broilers relative to Leghorns (Martinez-Lemus, L. A. et al., Am. J. Physiol. 277:R190-197 (1999); Martinez-Lemus, L. A. et al., Poult. Sci. 82:1957-1964 (2003)). Endothelium derived factors such as nitric oxide (NO), endothelin-1 (ET-1) and some eicosanoids have been shown to have vasoactive properties in chicken (Wideman, R. F., Jr. et al., Poult. Sci. 78:714-721 (1999); Villamor, E. et al., Am. J. Physiol. Regul. Integr. Comp. Physiol. 282:R917-927 (2002); Martinez-Lemus, L. A. et al., Poult. Sci. 82:1957-1964 (2003)). It was found that NO attenuates PH induced by hypoxia or endotoxin in experiments employing nitric oxide synthase inhibitors and supplementation of diet with 1-arginine, a precursor of NO (Wideman, R. F., Jr. et al., Poult. Sci. 74:323-330 (1995); Odom, T. W. et al., Poult. Sci. 83:835-841 (2004); Wideman, R. F. et al. Poult. Sci. 83:485-494 (2004)). ET-1 has been shown to constrict chicken pulmonary arteries (Martinez-Lemus, L. A. et al., Poult. Sci. 82:1957-1964 (2003)). Examination of the eicosanoids has focused on the actions of thromboxane and prostacyclin. Thromboxane has a vasoconstrictive effect in chicken pulmonary and cardiac microvessels (Wideman, R. F., Jr. et al., Poult. Sci. 78:714-721 (1999); Wideman, R. F., Jr. et al., Poult. Sci. 80:647-655 (2001)). However, cyclooxygenase inhibitors have failed to produce an effect on hypoxia-induced hypertension or isolated pulmonary coronary artery rings (Wideman, R. F., Jr. et al., Poult. Sci. 78:714-721 (1999); Villamor, E. et al., Am. J. Physiol. Regul. Integr. Comp. Physiol. 282:R917-927 (2002); Odom, T. W. et al., Poult. Sci. 83:835-841 (2004)). Taken together these results suggest that blood pressure regulation in chicken may have similarities to that of mammals.

In mammals, the epoxyeicosatrienoic acids (the EETs) are paracrine and autocrine signaling molecules involved in the regulation of vascular homeostasis, blood pressure and inflammation (Roman, R. J. Physiol. Rev. 82:131-185 (2002)). They are produced from arachidonic acid by cytochrome P450 enzymes in the endothelium of lung, cardiac, and renal microvessels (Rosolowsky, M. et al. Am. J. Physiol. 264:H327-335 (1993); Zou, A. P. et al., Am. J. Physiol. 270:F822-832 (1996); Gebremedhin, D. et al., J. Vasc. Res. 35:274-284 (1998)). They have been shown to have vasodilatory actions in renal and cardiac microvessels through activation of large conductance Ca²⁺-activated K⁺ channels (Zou, A. P. et al., Am. J. Physiol. 270:F822-832 (1996); Zhang, Y. et al., Am. J. Physiol. Heart Circ. Physiol. 280:H2430-2440 (2001); Boardman, P. E. et al., Curr. Biol. 12:1965-1969 (2002)). The EETs also have vasodilatory effects in bronchial smooth muscle (Zeldin, D. C. et al., J. Clin. Invest. 95:2150-2160 (1995)). In general, the EETs display vasodilatory effects and appear to function as endogenous anti-inflammatory and hypotensive agents in most vascular beds.

The mammalian soluble epoxide hydrolase (sEH) catalyzes the hydrolysis of aliphatic epoxides such as the EETs to their corresponding diols, the dihydroxyeicosatrienoic acids (DHETs) (Zeldin, D. C. et al., J. Biol. Chem. 268:6402-6407 (1993)). This converts the EETs into compounds that resist incorporation into lipid bilayers and can be excreted by the organism (Weintraub, N. L. et al., Am. J. Physiol. 277:H2098-2108 (1999)). sEH has been implicated in the metabolism of EETs in human vasculature (Fang, X. et al., Am. J. Physiol. Heart Circ. Physiol. 287:H2412-2420 (2004)). It has also been shown to have a hypotensive effect in porcine coronary endothelial cells using an sEH inhibitor (Fang, X. et al., J. Biol. Chem. 276:14867-14874 (2001)). In vivo experiments employing rat and mouse models have confirmed sEH's role in the regulation of blood pressure. Treatment with sEH inhibitors have shown that the enzyme mediates blood pressure in rat models of hypertension (Yu, Z. et al., Circ. Res. 87:992-998 (2000); Imig, J. D. et al., Hypertension 39:690-694 (2002)). Recently, sEH inhibitors have been shown to reduce lung inflammation induced by tobacco smoke in a spontaneous hypertensive rat model (Smith, K. R. et al., Proc. Natl. Acad. Sci. USA 102:2186-2191 (2005)).

Chickens produce the EETs, as well as the EET hydrolysis products, the DHETs (Nakai, K. et al., J. Biol. Chem. 267:19503-19512 (1992)). Herein is shown the amino acid and nucleic acid sequences of a sEH homologue in chicken with epoxide hydrolase activity surprisingly similar to the mammalian enzymes. There remains a need for the prophylactic and therapeutic amelioration, inhibition or prevention of PHS in avians, for example, chickens and other agriculturally raised avians. The present invention addresses this and other needs.

### BRIEF SUMMARY OF THE INVENTION

In a first aspect, the present invention provides methods of inhibiting or preventing pulmonary hypertension syndrome or a symptom thereof in an avian subject in need thereof, said method comprising administering to said avian subject an effective amount of an inhibitor of soluble epoxide hydrolase (“sEH”), thereby inhibiting or preventing pulmonary hypertension syndrome or said symptom thereof in said avian subject.

In some embodiments, the sEH inhibitor is one or more compounds from Table 3, e.g. selected from the group consisting of compound numbers 700, 1515, 1138, 1271, 1272, 1285, 1289, 1302, 1308, 1270, 1318, 941, 982, 983, 909, 861 and 863. In some embodiments, the sEH inhibitor has an IC50 for an avian (e.g. chicken) sEH of about 8 nM or less, for example, an IC50 of about 7 nM, 6 nM, 5 nM, 4 nM, 3 nM, 2 nM, 1 nM, 0.5 nM or less.

In some embodiments, the methods further comprise co-administering a cis-epoxyeicosantrienoic acid (“EET”).

In some embodiments, the avian subject is of the Subclass Neognathae or of the Order Galliformes. In some embodiments, the avian subject is of the Family Phasianidae. In some embodiments, the avian subject is a chicken (Gallus).

In some embodiments, the inhibited or prevented symptom is pulmonary hypertension and/or right-sided congestive heart failure and/or hypoxemia.

In a further aspect, the invention provides a nucleic acid encoding a soluble epoxide hydrolase having at least 95% nucleic acid sequence identity to the nucleic acid sequence depicted in FIG. 1.

In a related aspect, the invention provides a soluble epoxide hydrolase having at least 95% amino acid sequence identity to the amino acid sequence depicted in FIG. 1.

### BRIEF DESCRIPTION OF THE DRAWINGS

FIG. 1 illustrates nucleotide sequence and translated protein sequence of the chicken sEH cDNA. This DNA sequence has accession # DQ120010 in the Genbank database.

FIG. 2 illustrates an alignment of the chicken sEH with human, mouse and frog sEH. The mammalian epoxide hydrolase “catalytic triad” residues are marked by arrows. Residues that polarize the epoxide moiety of the epoxide hydrolase substrate are marked by circles. The catalytic nucleophile of the sEH phosphatase activity is marked by a triangle.

FIG. 3 illustrates SDS-PAGE analysis of recombinant chicken sEH purification. Samples were run on a Novex precast 12% Tris-Glycine gel (Invitrogen, Carlsbad, Calif.), and stained with Coomassie Brilliant Blue. Lane 1:1 μg of 250 mM imidazole eluant. Lane 2: 7 μg of 50 mM imidazole wash. Lane 3: 30 μg of unbound fraction. Lane 4: 30 μg of 100,000 g supernatant. Lane 5: 5 μL of SeeBlue Plus 2 (Invitrogen, Carlsbad, Calif.) molecular weight marker.

FIG. 4 illustrates IC50 values for the urea based inhibitors N-cyclohexyl-N′-ethylurea (CEU), N,N′-dicyclohexylurea (DCU), N-cyclohexyl-N′-dodecylurea (CDU), N-adamantyl-N′-cyclohexylurea (ACU), and 12-(3-adamantane-1-yl-ureido)-dodecanoic acid (AUDA). Recombinant chicken sEH was partially purified as described. Assay conditions are described in the Materials and Methods section. Error bars represent the standard deviation.

FIG. 5 illustrates IC50 values for soluble epoxide hydrolase inhibitors screened against chicken sEH.

### DETAILED DESCRIPTION OF THE INVENTION

### Introduction

EETs and sEHI for Prevention and/or Amelioration of Avian Pulmonary Hypertension Syndrome

Surprisingly, it has now been discovered that avian pulmonary hypertension syndrome can be inhibited or even reversed by the use of inhibitors of the avian homologue of soluble epoxide hydrolase (“sEH”; inhibitors of this enzyme are sometimes referred to herein as “sEHI”). The mammalian sEH plays a role in the regulation of blood pressure and vascular homeostasis through its hydrolysis of the endothelial derived messenger molecules, the epoxyeicosatrienoic acids. The present application provides the cloning and expression of a soluble epoxide hydrolase homologue from chicken liver. The resulting 63 kDa protein has a PI of 6.1. The recombinant enzyme displayed epoxide hydrolase activity when assayed with [³H]-trans-1,3-diphenylpropene oxide (t-DPPO), as well as trans-9,10-epoxystearate and the cis-8,9-, 11,12-, and 14,15-epoxyeicosatrienoic acids. The chicken enzyme displayed a lower kcat/Km ratio for t-DPPO than mammalian enzymes. The enzyme was sensitive to urea-based inhibitors developed for mammalian soluble epoxide hydrolase. Therefore, inhibitors of soluble epoxide hydrolase find use to treat conditions where endothelial derived vasodilation is believed to be impaired, for example avian pulmonary hypertension syndrome.

### DEFINITIONS

Units, prefixes, and symbols are denoted in their Système International de Unites (SI) accepted form. Numeric ranges are inclusive of the numbers defining the range. Unless otherwise indicated, nucleic acids are written left to right in 5′ to 3′ orientation; amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety. Terms not defined herein have their ordinary meaning as understood by a person of skill in the art.

“cis-Epoxyeicosatrienoic acids” (“EETs”) are biomediators synthesized by cytochrome P450 epoxygenases. As discussed further in a separate section below, while the use of unmodified EETs is the most preferred, derivatives of EETs, such as amides and esters (both natural and synthetic), EETs analogs, and EETs optical isomers can all be used in the methods of the invention, both in pure form and as mixtures of these forms. For convenience of reference, the term “EETs” as used herein refers to all of these forms unless otherwise required by context.

“Epoxide hydrolases” (“EH;” EC 3.3.2.3) are enzymes in the alpha beta hydrolase fold family that add water to 3-membered cyclic ethers termed epoxides.

“Soluble epoxide hydrolase” (“sEH”) is an epoxide hydrolase which in mammalian endothelial and smooth muscle cells has been shown to convert EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The nucleic acid sequence of human sEH is published as GenBank accession number L05779. The evolution and nomenclature of the gene is discussed in Beetham et al., DNA Cell Biol. 14(1):61-71 (1995). Mammalian soluble epoxide hydrolase represents a single highly conserved gene product with over 90% homology between rodent and human (Arand et al., FEBS Lett., 338:251-256 (1994)). As noted herein, it has now been discovered that an avian soluble epoxide hydrolase exists.

Unless otherwise specified, as used herein, the term “sEH inhibitor” (also abbreviated as “sEHI”) refers to an inhibitor of an avian sEH, or an sEH of an avian subject treated by the present methods. Preferably, the inhibitor does not also inhibit the activity of microsomal epoxide hydrolase by more than 25% at concentrations at which the inhibitor inhibits sEH by at least 50%, and more preferably does not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor. In some embodiments, the sEHIs inhibit sEH in a standard in vitro assay with an IC50 concentration of 500 μM or less, for example, 100 μM, 50 μM, 10 μM, 1 μM, or less, with each succeeding lower IC50 being more preferred. In some embodiments, the sEHIs inhibit sEH in a standard in vitro assay with an IC50 concentration that is in the nanomolar range, for example, 100 nM, 50 nM, 10 nM, or less.

As used herein, the terms “subject” or “patient” refers to an avian animal, i.e., an animal of the Class Aves, and more particularly of the Subclass Neognathae or of the Order Galliformes. In some embodiments, a subject is an avian within the Family Phasianidae, for example, turkeys (Meleagris), Phasianinae, including partridge, peafowl, pheasant, quail, and chicken. In some embodiments, the subject is a chicken (Gallus), for example, a broiler or leghorn chicken.

The term “avian” refers to animals relating to, or derived from birds (i.e., belonging to the class Aves). Birds are bipedal, warm-blooded, egg-laying vertebrates characterized primarily by feathers, forelimbs modified as wings, and hollow bones. Included in this definition are poultry and other avian species held captive for agricultural and/or breeding purposes (e.g., chickens, turkeys, geese, swan, ducks, loon, partridge, pheasant, grouse, emu, quail, ostrich, peacock, and related species).

“Pulmonary Hypertension Syndrome” or “PHS” refers to a number of conditions including right-sided congestive heart failure, hypoxemia, pulmonary hypertension (PH) and acites that can occur in avians, particularly poultry and other avian species held captive for agricultural and/or breeding purposes (e.g., chickens, turkeys, geese, swan, ducks, loon, partridge, grouse, emu, ostrich, peacock, and related species). PHS is reviewed, for example, in Wideman, R. F. Avian Poult. Rev. 11:21-43 (2000).

With respect to PHS, “inhibiting” or “ameliorating” means that the signs or symptoms of pulmonary hypertension syndrome are reduced or eliminated, or that the duration of such symptoms is reduced, or both. Inhibiting or ameliorating also means (i) the prevention of the development of the condition in a subject at risk thereof or (ii) the reversal of the signs or symptoms of PHS. To determine the extent of inhibition, comparisons can be made between treated and untreated subjects or between subjects before and after treatment.

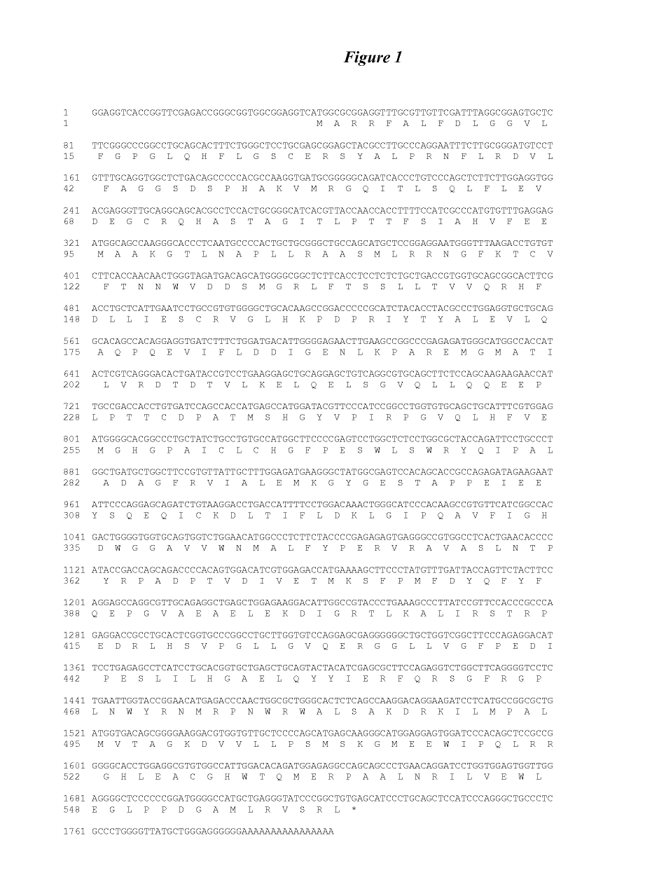

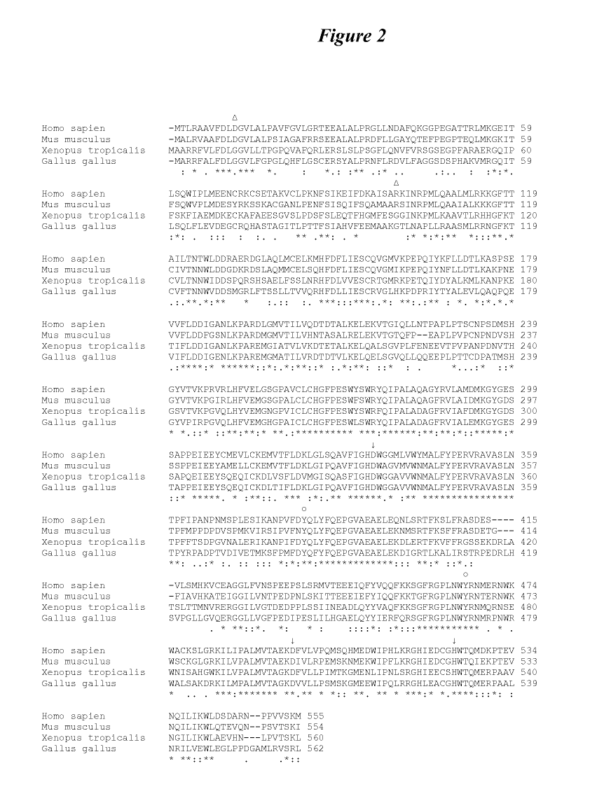

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any of the sequences in FIGS. 1 and 2), when compared and aligned for maximum correspondence over a comparison window or designated region) as measured using a BLAST or BLAST 2.0 sequence comparison algorithms with default parameters described below, or by manual alignment and visual inspection (see, e.g. NCBI web site or the like). Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequence.

For sequence comparison, typically one sequence acts as a reference sequence, to which test sequences are compared (here, a chicken sEH sequence). When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A preferred example of algorithm that is suitable for determining percent sequence identity and sequence similarity are the BLAST algorithms, which are described in Altschul et al., Nuc. Acids Res. 25:3389-3402 (1977) and Altschul et al., J. Mol. Biol. 215:403-410 (1990), respectively. BLAST software is publicly available through the National Center for Biotechnology Information on the worldwide web at ncbi.nlm.nih.gov/. Both default parameters or other non-default parameters can be used. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

Subjects that Benefit from the Present Methods

Avian subjects that benefit from the present methods are those that have or are at risk of developing the symptoms or signs of avian pulmonary hypertension syndrome. It is demonstrated herein that surprisingly chickens express soluble epoxide hydrolase. Having demonstrated that chickens express a soluble epoxide hydrolase, it is expected that other avian species will also express a soluble epoxide hydrolase. Further, having demonstrated that sEH inhibitors that inhibit human sEH can also effectively inhibit the enzymatic action of chicken sEH, it is expected that the enzymatic activity of sEH in other avian species can also be inhibited by sEH inhibitors.

Accordingly, the population that can benefit from the present methods include any avian animal, and more particularly of the Subclass Neognathae or of the Order Galliformes. In some embodiments, a subject is an avian within the Family Phasianidae, for example, turkeys (Meleagris), Phasianinae, including partridge, peafowl, pheasant, quail, and chicken (Gallus). In some embodiments, the subject is a chicken (Gallus), for example, a broiler or leghorn chicken. The methods find use in treating avian species held captive for agricultural and/or breeding purposes (e.g., chickens, turkeys, geese, swan, ducks, loon, partridge, pheasant, grouse, emu, quail, ostrich, peacock, and related species).

### Inhibitors of Soluble Epoxide Hydrolase

Scores of sEH inhibitors are known, of a variety of chemical structures. Derivatives in which the urea, carbamate, or amide pharmacophore (as used herein, “pharmacophore” refers to the section of the structure of a ligand that binds to the sEH) is covalently bound to both an adamantane and to a 12 carbon chain dodecane are particularly useful as sEH inhibitors. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-admamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include:

12-(3-Adamantan-1-yl-ureido)dodecanoic acid (AUDA),

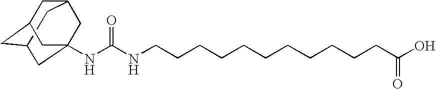

12-(3-Adamantan-1-yl-ureido)dodecanoic acid butyl ester (AUDA-BE),

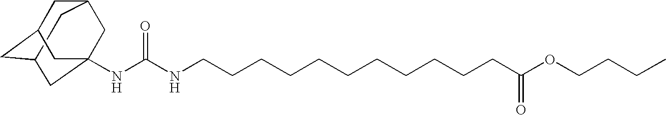

Adamantan-1-yl-3-{5-[2-(2-ethoxyethoxy)ethoxy]pentyl}urea (compound 950), and

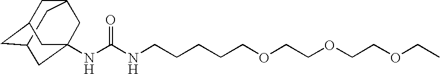

N-(1-acetylpiperidin-4-yl)-N′-(adamant-1-yl) urea (compound 1153).

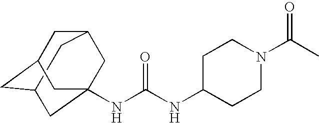

Further piperidine compounds that find use in the present methods include those disclosed in Jones, P. D., et al., Bioorg. Med. Chem. Lett. (2006) 16:5212-5216, incorporated herein by reference in its entirety. A number of other inhibitors which can be used in the methods and compositions of the invention are set forth in co-owned applications PCT/US2004/010298 and U.S. Published Patent Application Publication 2005/0026844.

U.S. Pat. No. 5,955,496 (the '496 patent) also sets forth a number of epoxide hydrolase inhibitors which can be use in the methods of the invention. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods of the invention are set forth in U.S. Pat. Nos. 6,150,415 (the '415 patent) and 6,531,506 (the '506 patent). Two preferred classes of inhibitors of the invention are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty inhibitors of Formula 2, which were shown to inhibit human sEH at concentrations as low as 0.1 μM. Any particular inhibitor can readily be tested to determine whether it will work in the methods of the invention by standard assays, such as that set forth in the Examples, below. Esters and salts of the various compounds discussed above or in the cited patents, for example, can be readily tested by these assays for their use in the methods of the invention.

As noted above, chalcone oxides can serve as an alternate substrate for the enzyme. While chalcone oxides have half lives which depend in part on the particular structure, as a group the chalcone oxides tend to have relatively short half lives (a drug's half life is usually defined as the time for the concentration of the drug to drop to half its original value. See, e.g., Thomas, G., Medicinal Chemistry: an introduction, John Wiley & Sons Ltd. (West Sussex, England, 2000)). Since the uses of the invention contemplate inhibition of sEH over periods of time which can be measured in days, weeks, or months, chalcone oxides, and other inhibitors which have a half life whose duration is shorter than the practitioner deems desirable, are preferably administered in a manner which provides the agent over a period of time. For example, the inhibitor can be provided in materials that release the inhibitor slowly, including materials that release the inhibitor in or near the kidney, to provide a high local concentration. Methods of administration that permit high local concentrations of an inhibitor over a period of time are known, and are not limited to use with inhibitors which have short half lives although, for inhibitors with a relatively short half life, they are a preferred method of administration.

In addition to the compounds in Formula 1 of the '506 patent, which interact with the enzyme in a reversible fashion based on the inhibitor mimicking an enzyme-substrate transition state or reaction intermediate, one can have compounds that are irreversible inhibitors of the enzyme. The active structures such as those in the Tables or Formula 1 of the '506 patent can direct the inhibitor to the enzyme where a reactive functionality in the enzyme catalytic site can form a covalent bond with the inhibitor. One group of molecules which could interact like this would have a leaving group such as a halogen or tosylate which could be attacked in an SN2 manner with a lysine or histidine. Alternatively, the reactive functionality could be an epoxide or Michael acceptor such as an α/β-unsaturated ester, aldehyde, ketone, ester, or nitrile.

Further, in addition to the Formula 1 compounds, active derivatives can be designed for practicing the invention. For example, dicyclohexyl thio urea can be oxidized to dicyclohexylcarbodiimide which, with enzyme or aqueous acid (physiological saline), will form an active dicyclohexylurea. Alternatively, the acidic protons on carbamates or ureas can be replaced with a variety of substituents which, upon oxidation, hydrolysis or attack by a nucleophile such as glutathione, will yield the corresponding parent structure. These materials are known as prodrugs or protoxins (Gilman et al., The Pharmacological Basis of Therapeutics, 7th Edition, MacMillan Publishing Company, New York, p. 16 (1985)) Esters, for example, are common prodrugs which are released to give the corresponding alcohols and acids enzymatically (Yoshigae et al., Chirality, 9:661-666 (1997)). The drugs and prodrugs can be chiral for greater specificity. These derivatives have been extensively used in medicinal and agricultural chemistry to alter the pharmacological properties of the compounds such as enhancing water solubility, improving formulation chemistry, altering tissue targeting, altering volume of distribution, and altering penetration. They also have been used to alter toxicology profiles.

In some embodiments, the sEH inhibitor is one or more compounds from Table 3, e.g. selected from the group consisting of compound numbers 700, 1515, 1138, 1271, 1272, 1285, 1289, 1302, 1308, 1270, 1318, 941, 982, 983, 909, 861 and 863. In some embodiments, the sEH inhibitor has an IC50 for an avian (e.g. chicken) sEH of about 8 nM or less, for example, an IC50 of about 7 nM, 6 nM, 5 nM, 4 nM, 3 nM, 2 nM, 1 nM, 0.5 nM or less.

In some embodiments, the sEH inhibitor is triclocarban (“TCC”).

In some embodiments, the sEH inhibitor is a “soft-drug” or “soft-compound,” in that it is metabolized to a water soluble compound by the avian subject and excreted from the animal rather than retained in the tissues of the avian subject.

In some embodiments, the sEH inhibitor is administered as a prodrug. There are many prodrugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al., Journal of Agricultural and Food Chemistry, 21(5):747-751 (1973); Fahmy et al, Journal of Agricultural and Food Chemistry, 26(3):550-556 (1978); Jojima et al., Journal of Agricultural and Food Chemistry, 31(3):613-620 (1983); and Fahmy et al., Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors of the invention mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, sEH inhibition can include the reduction of the amount of sEH. As used herein, therefore, sEH inhibitors can therefore encompass nucleic acids that inhibit expression of a gene encoding sEH. Many methods of reducing the expression of genes, such as reduction of transcription and siRNA, are known, and are discussed in more detail below.

Preferably, the inhibitor inhibits sEH without also significantly inhibiting microsomal epoxide hydrolase (“mEH”). Preferably, at concentrations of 500 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 500 μM. Inhibitors with IC₅₀s of less than 500 μM are preferred, with IC₅₀s of less than 100 μM being more preferred and, in order of increasing preference, an IC50 of 50 μM, 40 μM, 30 μM, 25 μM, 20 μM, 15 μM, 10 μM, 5 μM, 3 μM, 2 μM, 1 μM or even less being still more preferred. Assays for determining sEH activity are known in the art and described elsewhere herein.

### EETs

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into dihydroxyeicosatrienoic acids (“DHETs”) is reduced.

It has long been believed that EETs administered systemically would be hydrolyzed too quickly by endogenous sEH to be helpful. In the only prior report of EETs administration of which we are aware, EETs were administered by catheters inserted into mouse aortas. The EETs were infused continuously during the course of the experiment because of concerns over the short half life of the EETs. See, Liao and Zeldin, International Publication WO 01/10438 (hereafter “Liao and Zeldin”). It also was not known whether endogenous sEH could be inhibited sufficiently in body tissues to permit administration of exogenous EET to result in increased levels of EETs over those normally present. Further, it was thought that EETs, as epoxides, would be too labile to survive the storage and handling necessary for therapeutic use.

In studies from the laboratory of one of the present inventors, however, it has been shown that systemic administration of EETs in conjunction with inhibitors of sEH had better results than did administration of sEH inhibitors alone. EETs were not administered by themselves in these studies since it was anticipated they would be degraded too quickly to have a useful effect. Additional studies from the laboratory of one of the present inventors have now shown, however, that administration of EETs by themselves has had therapeutic effect. Without wishing to be bound by theory, it is surmised that the exogenous EET overwhelms endogenous sEH, and allows EETs levels to be increased for a sufficient period of time to have therapeutic effect. Thus, EETs can be administered without also administering an sEHI to provide a therapeutic effect. Moreover, we have found that EETs, if not exposed to acidic conditions or to sEH are stable and can withstand reasonable storage, handling and administration.

In short, sEHI, EETs, or co-administration of sEHIs and of EETs, can be used to inhibit the development of, or reverse the presence of, cardiac hypertrophy, of dilated cardiomyopathy, and of atrial and of ventricular fibrillation. In some embodiments, one or more EETs are administered to the patient without also administering an sEHI. In some embodiments, one or more EETs are administered shortly before or concurrently with administration of an sEH inhibitor to slow hydrolysis of the EET or EETs. In some embodiments, one or more EETs are administered after administration of an sEH inhibitor, but before the level of the sEHI has diminished below a level effective to slow the hydrolysis of the EETs.

EETs useful in the methods of the present invention include 14,15-EET, 8,9-EET and 11,12-EET, and 5,6 EETs. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

If desired, EETs, analogs, or derivatives that retain activity can be used in place of or in combination with unmodified EETs. Liao and Zeldin, supra, define EET analogs as compounds with structural substitutions or alterations in an EET, and include structural analogs in which one or more EET olefins are removed or replaced with acetylene or cyclopropane groups, analogs in which the epoxide moiety is replaced with oxitane or furan rings and heteroatom analogs. In other analogs, the epoxide moiety is replaced with ether, alkoxides, difluorocycloprane, or carbonyl, while in others, the carboxylic acid moiety is replaced with a commonly used mimic, such as a nitrogen heterocycle, a sulfonamide, or another polar functionality. In preferred forms, the analogs or derivatives are relatively stable as compared to an unmodified EET because they are more resistant than an EET to sEH and to chemical breakdown. “Relatively stable” means the rate of hydrolysis by sEH is at least 25% less than the hydrolysis of the unmodified EET in a hydrolysis assay, more preferably 50% or more lower than the rate of hydrolysis of an unmodified EET. Liao and Zeldin show, for example, episulfide and sulfonamide EETs derivatives. Amide and ester derivatives of EETs and that are relatively stable are preferred embodiments. In preferred forms, the analogs or derivatives have the biological activity of the unmodified EET regioisomer from which it is modified or derived in reducing cardiac hypertrophy, dilated cardiomyopathy, or arrhythmia. Whether or not a particular EET analog or derivative has the biological activity of the unmodified EET can be readily determined by using it in the assays described in the Examples. As mentioned in the Definition section, above, for convenience of reference, the term “EETs” as used herein refers to unmodified EETs, and EETs analogs and derivatives unless otherwise required by context.

In some embodiments, the EET or EETs are embedded or otherwise placed in a material that releases the EET over time. Materials suitable for promoting the slow release of compositions such as EETs are known in the art. Optionally, one or more sEH inhibitors may also be placed in the slow release material.

Conveniently, the EET or EETs can be administered orally. Since EETs are subject to degradation under acidic conditions, EETs intended for oral administration can be coated with a coating resistant to dissolving under acidic conditions, but which dissolve under the mildly basic conditions present in the intestines. Suitable coatings, commonly known as “enteric coatings” are widely used for products, which cause gastric distress or which would undergo degradation upon exposure to gastric acid. By using coatings with an appropriate dissolution profile, the coated substance can be released in a chosen section of the intestinal tract. Such coatings are commercially available from, for example, Rohm Specialty Acrylics (Rohm America LLC, Piscataway, N.J.) under the trade name “Eudragit®”. The choice of the particular enteric coating is not critical to the practice of the invention.

### Assays for Epoxide Hydrolase Activity

Any of a number of standard assays for determining epoxide hydrolase activity can be used to determine inhibition of sEH. For example, suitable assays are described in Gill, et al., Anal Biochem 131, 273-282 (1983); and Borhan, et al., Analytical Biochemistry 231, 188-200 (1995)). Suitable in vitro assays are described in Zeldin et al., J. Biol. Chem. 268:6402-6407 (1993). Suitable in vivo assays are described in Zeldin et al., Arch Biochem Biophys 330:87-96 (1996). Assays for epoxide hydrolase using both putative natural substrates and surrogate substrates have been reviewed (see, Hammock, et al. In: Methods in Enzymology, Volume III, Steroids and Isoprenoids, Part B, (Law, J. H. and H. C. Rilling, eds. 1985), Academic Press, Orlando, Fla., pp. 303-311 and Wixtrom et al., In: Biochemical Pharmacology and Toxicology, Vol. 1: Methodological Aspects of Drug Metabolizing Enzymes, (Zakim, D. and D. A. Vessey, eds. 1985), John Wiley & Sons, Inc., New York, pp. 1-93. Several spectral based assays exist based on the reactivity or tendency of the resulting diol product to hydrogen bond (see, e.g., Wixtrom, supra, and Hammock. Anal. Biochem. 174:291-299 (1985) and Dietze, et al. Anal. Biochem. 216:176-187 (1994)).

The enzyme also can be detected based on the binding of specific ligands to the catalytic site which either immobilize the enzyme or label it with a probe such as dansyl, fluoracein, luciferase, green fluorescent protein or other reagent. The enzyme can be assayed by its hydration of EETs, its hydrolysis of an epoxide to give a colored product as described by Dietze et al., 1994, supra, or its hydrolysis of a radioactive surrogate substrate (Borhan et al., 1995, supra). The enzyme also can be detected based on the generation of fluorescent products following the hydrolysis of the epoxide. Numerous method of epoxide hydrolase detection have been described (see, e.g., Wixtrom, supra).

The assays are normally carried out with a recombinant enzyme following affinity purification. They can be carried out in crude tissue homogenates, cell culture or even in vivo, as known in the art and described in the references cited above.

### Other Means of Inhibiting sEH Activity

Other means of inhibiting sEH activity or gene expression can also be used in the methods of the invention. For example, a nucleic acid molecule complementary to at least a portion of the avian sEH gene can be used to inhibit sEH gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science. 305(5688): 1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet. 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo. In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of sEH, siRNAs to the gene encoding sEH can be specifically designed using computer programs. The cloning, sequence, and accession numbers of the human sEH sequence are set forth in Beetham et al., Arch. Biochem. Biophys. 305(1):197-201 (1993). The amino acid sequence of human sEH and the nucleotide sequence encoding that amino acid sequence are set forth in U.S. Pat. No. 5,445,956.

A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/mai) and, to academic and non-profit researchers, from the Whitehead Institute for Biomedical Research on the internet by entering “http://” followed by “jura.wi.mit.edu/pubint/http://iona.wi.mit.edu/siRNAext/.”

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplar vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human HI. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO: ______). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence.

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the antisense sequence is substantially complementary to the target sequence. In certain embodiments, the antisense sequence is exactly complementary to the target sequence. The antisense polynucleotides may also include, however, nucleotide substitutions, additions, deletions, transitions, transpositions, or modifications, or other nucleic acid sequences or non-nucleic acid moieties so long as specific binding to the relevant target sequence corresponding to the sEH gene is retained as a functional property of the polynucleotide. In one embodiment, the antisense molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program on the internet which can be found by entering http://, followed by biotools.idtdna.com/antisense/AntiSense.aspx, which will provide appropriate antisense sequences for nucleic acid sequences up to 10,000 nucleotides in length.

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) a sEH gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found by entering “www.” followed by “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol. Chem. 279(40):42230-9 (2004); Ying and Lin, Biochem Biophys Res Commun. 326(3):515-20 (2005).

### Therapeutic Administration

EETs and inhibitors of sEH can be prepared and administered in a wide variety of oral, parenteral and aerosol formulations. In some embodiments, compounds for use in the methods of the present invention can be administered orally or by injection, that is, intravenously, intramuscularly, intracutaneously, subcutaneously, or intraperitoneally. The sEH inhibitor or EETs, or both, can also be administered by inhalation, for example, through the beak or mouth. Additionally, the sEH inhibitors, or EETs, or both, can be administered transdermally. In some embodiments, the sEH inhibitors, or EETs, or both, are mixed into the avian feed or water. Accordingly, the methods of the invention permit administration of pharmaceutical compositions comprising a pharmaceutically acceptable carrier or excipient and either a selected inhibitor or a pharmaceutically acceptable salt of the inhibitor.

For preparing pharmaceutical compositions from sEH inhibitors, or EETs, or both, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired. The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution. Transdermal administration can be performed using suitable carriers. If desired, apparatuses designed to facilitate transdermal delivery can be employed. Suitable carriers and apparatuses are well known in the art, as exemplified by U.S. Pat. Nos. 6,635,274, 6,623,457, 6,562,004, and 6,274,166.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The term “unit dosage form”, as used in the specification, refers to physically discrete units suitable as unitary dosages for animals, each unit containing a predetermined quantity of active material calculated to produce the desired pharmaceutical effect in association with the required pharmaceutical diluent, carrier or vehicle. The specifications for the novel unit dosage forms of this invention are dictated by and directly dependent on (a) the unique characteristics of the active material and the particular effect to be achieved and (b) the limitations inherent in the art of compounding such an active material for use in animals, as disclosed in detail in this specification, these being features of the present invention.

A therapeutically effective amount of the sEH inhibitor, or EETs, or both, is employed in inhibiting or preventing avian pulmonary hypertension syndrome. The dosage of the specific compound for treatment depends on many factors that are well known to those skilled in the art. They include for example, the route of administration and the potency of the particular compound. An exemplary dose is from about 0.001 μg/kg to about 100 mg/kg body weight of the bird, for example, about 0.01 μg/kg, 0.1 μg/kg, 1.0 μg/kg, 10 μg/kg, 100 μg/kg, 1 mg/kg or 10 mg/kg.

The sEH inhibitors, or EETs, or both can be delivered to a subject as often as needed, for prophylactic or therapeutic purposes. For example, the sEH inhibitors, or EETs, or both can be administered once a day, once a week, once a month, or more or less often, as needed. In some embodiments, the sEH inhibitors, or EETs, or both can be administered therapeutically for a duration of time until a desired effect is achieved (e.g., on the order of weeks or months) and then continued for maintenance or prophylactic purposes for as long as is desired or needed (e.g., on the order of weeks, months, or years, or for the life-span of the animal).

EETs are unstable in acidic conditions, and can be converted to DHET. To avoid conversion of the EETs to DHET under acidic conditions in the avian proventriculus, EETs can be administered intravenously, by injection, or by aerosol. EETs intended for oral administration can be encapsulated in a coating that protects the EETs during passage through acid conditions in the proventriculus and gizzard. For example, the EETs can be provided with a so-called “enteric” coating, or embedded in a formulation. Such enteric coatings and formulations are well known in the art. In some formulations, the EETs, or a combination of the EETs and an sEH inhibitor are embedded in a slow-release formulation to facilitate administration of the agents over time.

In another set of embodiments, an sEH inhibitor, one or more EETs, or both an sEH inhibitor and an EET are administered by delivery to the air sacs or to the lungs. Air sac and pulmonary delivery are considered to be ways drugs can be rapidly introduced into an organism. Devices for delivering substances (e.g. gases) to avian lungs are known in the art. Environmental atmospheric devices can be used to deliver either an aerosol of an therapeutically active agent in a solution, or a dry powder of the agent via inhalation. To aid in providing reproducible dosages of the agent, dry powder formulations can include substantial amounts of excipients, such as polysaccharides, as bulking agents. Dosages can be further controlled by limiting the number of birds in a finite closed space subject to atmospheric delivery through inhalation.

Detailed information about the delivery of therapeutically active agents in the form of aerosols or as powders is available in the art. For example, the Center for Drug Evaluation and Research (“CDER”) of the U.S. Food and Drug Administration provides detailed guidance in a publication entitled: “Guidance for Industry: Nasal Spray and Inhalation Solution, Suspension, and Spray Drug Products—Chemistry, Manufacturing, and Controls Documentation” (Office of Training and Communications, Division of Drug Information, CDER, FDA, July 2002). This guidance is available in written form from CDER, or can be found on-line by entering “http://www.” followed by “fda.gov/cder/guidance/4234fnl.htm”. The FDA has also made detailed draft guidance available on dry powder inhalers and metered dose inhalers. See, Metered Dose Inhaler (MDI) and Dry Powder Inhaler (DPI) Drug Products—Chemistry, Manufacturing, and Controls Documentation, 63 Fed. Reg. 64270, (November 1998). This information can be readily adapted for use in avian species, such as chickens and turkeys.

In some aspects of the invention, the sEH inhibitor, EET, or combination thereof, is dissolved or suspended in a suitable solvent, such as water, ethanol, or saline, and administered by nebulization. A nebulizer produces an aerosol of fine particles by breaking a fluid into fine droplets and dispersing them into a flowing stream of gas. Medical nebulizers are designed to convert water or aqueous solutions or colloidal suspensions to aerosols of fine, inhalable droplets that can enter the lungs of a subject during inhalation and deposit on the surface of the respiratory airways. Typical pneumatic (compressed gas) medical nebulizers develop approximately 15 to 30 microliters of aerosol per liter of gas in finely divided droplets with volume or mass median diameters in the respirable range of 2 to 4 micrometers. Predominantly, water or saline solutions are used with low solute concentrations, typically ranging from 1.0 to 5.0 mg/mL. Methods for administering aerosols to birds are taught in, e.g., U.S. Pat. Nos. 5,109,797, 6,725,859, and 6,904,912.

Nebulizers for delivering an aerosolized solution to the lungs are commercially available from a number of sources, including the AERx™ (Aradigm Corp., Hayward, Calif.) and the Acorn II® (Vital Signs Inc., Totowa, N.J.) and can be adapted for use by avian subjects as described in the patents referenced in the preceding paragraph.

Metered dose inhalers are also known and can be adapted for use with avian subjects. Breath actuated inhalers typically contain a pressurized propellant and provide a metered dose automatically when the patient's inspiratory effort either moves a mechanical lever or the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978; and 4,896,832.

The formulations may also be delivered using a dry powder inhaler (DPI), i.e., an inhaler device that utilizes the subject's inhaled breath as a vehicle to transport the dry powder drug to the lungs. Such devices are described in, for example, U.S. Pat. Nos. 5,458,135; 5,740,794; and 5,785,049. When administered using a device of this type, the powder is contained in a receptacle having a puncturable lid or other access surface, preferably a blister package or cartridge, where the receptacle may contain a single dosage unit or multiple dosage units.

Other dry powder dispersion devices for pulmonary administration of dry powders include those described in Newell, European Patent No. EP 129985; in Hodson, European Patent No. EP 472598, in Cocozza, European Patent No. EP 467172, and in Lloyd, U.S. Pat. Nos. 5,522,385; 4,668,281; 4,667,668; and 4,805,811. Dry powders may also be delivered using a pressurized, metered dose inhaler (MDI) containing a solution or suspension of drug in a pharmaceutically inert liquid propellant, e.g., a chlorofluorocarbon or fluorocarbon, as described in U.S. Pat. Nos. 5,320,094 and 5,672,581.

Without further elaboration, it is believed that one skilled in the art can, using the preceding description, practice the present invention to its fullest extent.

### EXAMPLES

### Example 1

The following example demonstrates the cloning and characterization of soluble epoxide hydrolase from chickens.

### Materials and Methods

Total RNA and cDNA Library Preparation

Liver (0.5 g) from a 6 to 8 wk male Cobb broiler chicken was homogenized in 7.0 mL TRIzol reagent (Invitrogen, Carlsbad, Calif.) with a Polytron grinder (Brinkmann Instruments, Westbury, N.Y.) rotating at 9,000 rpm for 1 min, then left at room temperature for 5 min. The sample was centrifuged 12,000 g for 15 min at 4° C., then 1.4 mL chloroform was added. The sample was left at room temperature for 5 min and then centrifuged 12,000 g for 15 min at 4° C. The upper phase was transferred to a new tube, and 3.5 mL isopropanol added. After a 10 min incubation at room temperature, the sample was spun 12,000 g for 10 min at 4° C. The supernatant was discarded and 7 mL of 75% ethanol added. The sample was vortexed 30 s and centrifuged at 7,500 g for 5 min at 4° C. This 75% ethanol wash was repeated one more time and then the pellet was allowed to air dry 10 min. The pellet was resuspended in DEPC treated ddH₂O. mRNA enrichment was performed using the Oligotex mRNA kit.² A first strand cDNA library was constructed using the Superscript First-Strand Synthesis System for RT-PCR.²

### 5′ and 3′ RACE Experiments and Polymerase Chain Reaction

5′ RACE experiment was performed on the total RNA sample with the 5′ RACE System for Rapid Amplification of cDNA Ends kit2 using the nested primers 5R1: 5′-CTGAAGCCAGACCTCTGGAA-3′, 5R2: 5′-CCGTGCAGGATGAGGCTCTCA GGAATGT-3′, and 5R3: 5′-CCCTCGCTCCTGGACACCAAGCA-3′. 3′ RACE experiment was performed on the total RNA sample with the 3′ RACE System for Rapid Amplification of cDNA Ends kit2 using the nested primers 3R1: 5′-AAGCCCTTATCC GTTCCACCCGCC-3′, 3R2: 5′-TGCTTGGTGTCCAGGAGCGAGGG-3′, and 3R3: 5′-ACATTCCTGAGAGCCTCATCCTGCAC-3′. Polymerase chain reaction was performed on the chicken liver cDNA library using the primers CHXF: 5′-GCGGCC GCATGGCGCGGAGGTTTGCGTTGTTC-3′ and CHXR: 5′-GCGGCCGCTCAC AGCCGGGATACCCTCAGCATG-3′ and Pfu polymerase (Stratagene, La Jolla, Calif.) according to standard technique (Sambrook, J. et al., Molecular Cloning: A Laboratory Manual. Cold Spring Harbor Laboratory Press, Cold Spring Harbor, N.Y. (2001)). The clone was inserted into the vector pCR-Blunt using the Zero Blunt PCR cloning kit.²

### Baculovirus Expression

Baculovirus construction was performed using the Bac-to-Bac Baculovirus Expression System (Invitrogen). Sf21 insect cells (Invitrogen) were used to amplify the virus. Baculovirus titer was determined using the BD BakPAK Baculovirus Rapid Titer Kit (BD Biosciences Clontech, Palo Alto, Calif.). A 100 mL culture of T. ni cells were infected at 0.1 MOI and incubated for 1 h at 28° C. then 400 mL of ESF921 media (Expression Systems, Woodland, Calif.) supplemented with 1× Penicillin-Streptomycin solution (Sigma-Aldrich, St. Louis, Mo.) was added to the infected cells and the culture was incubated for 72 h at 28° C.

### Protein Purification

Infected T. ni cells (250 mL) were pelleted and resuspended in phosphate buffer with 10 mM imidazole. The cells were homogenized with an Ultra-Turax T25 homogenizer (IKA Works, Wilmington, N.C.) at 17,500 rpm for three 30 s intervals, with 15 s rest on ice between each grinding. The homogenate was centrifuged at 100,000 g for 1 h at 4° C. Ni-NTA HisBind Resin (0.3 mL) (EMD Biosciences, Inc., Madison, Wis.) was rinsed with 25 mL 10 mM phosphate buffer pH 7.4. The supernatant was then gently mixed with the resin at 4° C. for 1 hr. The supernatant and resin mixture was poured into a 25 mL Bio-Rad disposable column (Bio-Rad Laboratories, Inc., Hercules, Calif.) and allowed to drain by gravity flow. The column was then washed with 45 mL phosphate buffer containing 60 mM imidazole at a rate of 1 mL/min. The bound protein was eluted with 5 mL phosphate buffer containing 250 mM imidazole at a rate of 1 mL/min. The eluant was concentrated to 1.5 mL on a 30 kDa cut Centricon centrifugation filtration device (Millipore, Billerica, Mass.). The concentrated sample was applied to a 5 mL desalting column (Amersham, Piscataway, N.J.) and eluted in 2 mL 25 mM Tris-HCL, pH 7.5. 100 μL aliquots were frozen in liquid nitrogen and stored at −80° C. for future use.

### Extract Preparation

Fresh chicken liver was obtained from a 3-wk-old male Cobb×Cobb broiler fed a corn/soy broiler starter diet that met NRC requirements. Liver was cut in small pieces and suspended in 40 mL 20 mM sodium phosphate buffer at 4° C. (pH 7.4) containing 5 mM of EDTA and 1 mM of PMSF and DTT. The suspension was homogenized with a Polytron grinder rotating at 9,000 rpm for 1 min. The homogenate was centrifuged at 10,000 g for 20 min at 4° C. The supernatant was then centrifuged at 100,000 g for 60 min at 4° C. The supernatant (the cytosol) was frozen at −80° C. until used as enzyme extract.

### Protein Analysis

Protein concentration measurements were made using the BCA assay (Pierce, Rockford, Ill.) with BSA fraction V protein (Sigma-Aldrich) to derive a standard curve. Polyacrylamide gel electrophoresis was performed using Novex precast polyacrylamide gels (Invitrogen) for both SDS-PAGE analysis and isoelectric focusing. SDS-PAGE gels were stained with Coomassie Brilliant Blue. Bands from isoelectric focusing gels (3 to 10 pH) were excised and tested for epoxide hydrolase activity using the radioactive epoxide hydrolase assay described below. Protein purity was estimated from a SDS-PAGE gel stained with Coomassie Brilliant Blue with the public domain ImageJ software v1.33 (on the World Wide Web at rsb.info.nih.gov/ij/).

### Radiotracer Based Epoxide Hydrolase Activity Assay

Epoxide hydrolase activity was measured using racemic [³H]-trans-1,3-diphenylpropene oxide (t-DPPO) as substrate (Borhan, B. et al., Anal. Biochem. 231:188-200 (1995)). t-DPPO was previously synthesized and purified Borhan, B. et al., Anal. Biochem. 231:188-200 (1995)). 1 μL of a 5 mM solution of [³H]t-DPPO in DMF was added to 100 μL of enzyme preparation in sodium phosphate buffer (0.1 M, pH 7.4) containing 0.1 mg/mL of BSA ([S]_{final}=50 μM). The enzyme was incubated at 30oC for 10 min, and the reaction quenched by addition of 60 μL of methanol and 200 μL of isooctane, which extracts the remaining epoxide from the aqueous phase. Extractions with 1-hexanol were performed in parallel to assess the possible presence of glutathione transferase activity which could also transform the substrate (Borhan, B. et al., Anal. Biochem. 231:188-200 (1995)). The activity was followed by measuring the quantity of radioactive diol formed in the aqueous phase using a scintillation counter (Wallac Model 1409, Gaithersburg, Md.). Assays were performed in triplicate.

### IC₅₀ Determination

The IC₅₀ values reported herein were determined using racemic [³H]-trans-1,3-diphenylpropene oxide (t-DPPO) as a substrate (Borhan, B. et al., Anal. Biochem. 231:188-200 (1995)). The inhibitors were synthesized as described (Morisseau, C. et al., Biochem. Pharmacol. 63:1599-1608 (2002)). Extracts of broiler hepatic cytosol or partially purified enzyme was diluted 50-fold in pH 7.4 0.1 M sodium phosphate buffer containing 0.1 mg/mL BSA, then incubated with the inhibitors for 5 min at 30° C. prior to substrate introduction. Prepared samples were incubated at 30° C. for 10 min and stopped as indicated above. Conditions used gave rates that were linear both with time and enzyme concentration. Assays were performed in triplicate. By definition, IC₅₀ is the concentration of inhibitor that reduces enzyme activity by 50%. IC₅₀ was determined by regression with a minimum of two points in the linear region of the curve on either side of the IC₅₀ (for a total of 5 points). The curve was generated from at least three separate studies conducted in triplicate in order to obtain the standard deviation given in the results section.

### Determination of Kinetic Parameters

A solution of [³H] t-DPPO (1 μL in DMF) was added to 100 μL of enzyme preparation in sodium phosphate buffer (0.1 M, pH 7.4) containing 0.1 mg/mL of BSA ([S]_{final}=50 μM). The Kₘ determination was performed by fitting the data to the Michaelis-Menten equation using the nonlinear regression algorithm in SigmaPlot (SPSS, Inc., Chicago, Ill.) with an R-squared value of at least 0.97. The standard deviation was generated by performing the experiment three separate times in triplicate.

### Synthesis and Purification of EET Regioisomers

The epoxyeicosatrienoic acid isomeric mixture (8,9-, 11,12- and 14,15-EET) was synthesized from arachidonic acid methyl ester by a previously described method (Newman, J. W. et al., J. Lipid Res. 43:1563-1578 (2002); Smith, K. R. et al., Proc. Natl. Acad. Sci. USA 102:2186-2191 (2005)). The mixture was separated to 14,15-EET fraction (t_{R} 24.7 min) and 8,9- and 11,12-EET mixture fraction (t_{R} 29.5 min) with reverse phase preparative HPLC (C18, 22×250 mm) at a flow rate of 18 mL/min (75% of solvent A in solvent B; solvent A: acetonitrile-water-methanol, 51:40:9 (v/v/v) with 0.01% formic acid, solvent B: acetonitrile-methanol, 85:15 (v/v) with 0.01% formic acid). The mixture of 8,9- and 11,12-EET was separated to 8,9-EET fraction and 11,12-EET fraction with normal phase preparative HPLC (silica, 22×250 mm) at a flow rate of 18 mL/min using 1% iso-propanol in n-hexane. Each fraction was finally purified with normal phase HPLC with same condition described in above to give pure each isomer (8,9-EET: t_{R} 17.3 min, 11,12-EET: t_{R} 13.5 min, 14,15-EET: t_{R} 11.2 min).

### Non-Radioactive Epoxide Hydrolase Assays

The trans-9,10-epoxystearate was purchased (Sigma-Aldrich). A 5 mM solution of each substrate was made in ethanol for the EETs and methanol for the epoxystearate. The substrate solution (1 μL) was added to 100 μL of enzyme preparation in sodium phosphate buffer (0.1 M, pH 7.4) containing 0.1 mg/mL of BSA ([S]_{final}=50 μM). The enzyme was incubated with the substrate at 30° C. for 10 min, and the reaction quenched by addition of 400 μL of methanol. The products were analyzed by HPLC-MS/MS as previously described (Newman, J. W. et al., J. Lipid Res. 43:1563-1578 (2002)) with the following exceptions. A 2.0×20 mm, 3-μm Luna C18 Mercury MS column (Phenomenex, Torrance, Calif.) was used with a 350 μL/min isocratic flow of 68:28:11 (vol/vol/vol) acetonitrile/water/methanol with 0.1% glacial acetic acid for 2.5 min. Assays were performed in triplicate.

### Phosphatase Assay

The phosphatase substrate, threo-9,10-phosphonooxy-hydroxy-octadecanoic acid was synthesized as previously described (Newman, J. W. et al., Proc. Natl. Acad. Sci. USA 100: 1558-1563 (2003)). The assay was performed and analyzed by HPLC-MS/MS as described (Newman, J. W. et al., Proc. Natl. Acad. Sci. USA 100:1558-1563 (2003)).

### Results and Discussion

This study reports the cloning, expression, and characterization of a homologue of soluble epoxide hydrolase in chicken. Sequence fragments homologous to reported mammalian cDNA sequences were discovered in two EST databases. Sequences corresponding to the N terminal region of mammalian sEH were found in the University of Delaware ChikEST database at “http://www. “followed by chickest.udel.edu”/(Clone JDs: pgf2n.pk002.g24, pgf2n.pk001.d21, and pg11n.pk005.113), while sequences corresponding to the C-terminal region of mammalian sEH were found in the Biotechnology and Biological Sciences Research Council ChikEST database (template ID: 341537.2) at “http://www” followed by “.chick.umist.ac.uk/” (Boardman, P. E. et al., Curr. Biol. 12:1965-1969 (2002)). Primers for 5′ and 3′ rapid amplification of cDNA ends (RACE) experiments were designed based on these EST sequences. The RACE experiments indicated that these fragments came from a single cDNA sequence. The primers CHXF and CHXR were designed based on the predicted 5′ and 3′ end of this cDNA sequence, and a 1686 base cDNA was cloned from chicken liver (FIG. 1).

This sequence was used to probe the first draft chicken genome assembly determined by whole genome shotgun at the Genome Sequencing Center at Washington University, St. Louis. The majority of the cDNA sequence is located on chromosome 3 of the chicken genome. The first 100 base pairs of the cDNA sequence are located in unplaced sequences of the chicken genome. These 100 bases are contiguous and so may comprise the first exon of the gene. The last 92 base pairs of the cDNA sequence were not located in this draft of the chicken genome. There is a gap in the genome downstream from the last predicted exon of the sEH homologue gene, and it is possible that the missing base pairs fall within this gap. This will be discussed further after a closer examination of the sequence identities between the translated sequence from chicken and reported mammalian sEH sequences.

The nucleotide sequence of this clone displays homology to mammalian and frog sEH sequences when aligned by LALIGN. It displays a 62.2% identity to human sEH, a 60.5% identity to mouse sEH and a 63.7% identity to frog sEH. The translated sequence is 51% identical to the human sequence, 50.8% identical to mouse sEH, and 62.3% identical to frog sEH. Alignment of the amino acid sequences reveals a number of important structural similarities between the chicken and the human enzyme (FIG. 2). The mammalian sEH epoxide hydrolase catalytic triad is composed of a catalytic nucleophile, a histidine and an orienting acid. In the human enzyme this triad is represented by Asp³³⁴, His⁵²³, and Asp⁴⁹⁵ (Morisseau, C. et al., Annu. Rev. Pharmacol. Toxicol. 45:311-333 (2005)). The chicken enzyme preserves the identity and spacing of these residues (marked with an arrow in FIG. 2). Two tyrosines (Tyr³⁸² and Tyr⁴⁶⁵) polarize the epoxide in the human enzyme (Morisseau, C. et al., Annu. Rev. Pharmacol. Toxicol. 45:311-333 (2005)). These residues and their approximate spacing are also conserved in the chicken enzyme as Tyr³⁸³ and Tyr⁴⁷¹ respectively (marked by the circle in FIG. 2).

In addition to this epoxide hydrolase catalytic site, there is a second catalytic site on the N terminal region of the mammalian enzyme which has been shown to display phosphatase activity (Cronin, A. et al., Proc. Natl. Acad. Sci. USA 100:1552-1557 (2003); Newman, J. W. et al., Proc. Natl. Acad. Sci. USA 100:1558-1563 (2003)). The crystal structure of human sEH has implicated Asp¹¹ and Arg⁹⁹ as having roles in this phosphatase activity through their involvement in the coordination of a magnesium atom in the active site (Gomez, G. A. et al., Biochemistry 43:4716-4723 (2004)). These residues are not conserved in the chicken enzyme (marked by the triangles in FIG. 2).

As mentioned above, the last 92 base pairs of the cDNA transcript were not found in the first draft of the chicken genome. This region of the transcript encodes for residues important for epoxide hydrolase activity in mammalian enzymes. Specifically, an enzyme lacking these residues would not possess the histidine which aligns with His⁵²³ in the mouse sequence. Mutation of this residue in the mouse sEH abolishes epoxide hydrolase activity (Pinot, F. et al., J. Biol. Chem. 270:7968-7974 (1995)). The recombinant protein possesses epoxide hydrolase activity, providing evidence that the transcript represents the correct sequence of the chicken sEH homolog, and was not the result errors introduced during cloning.

Both the specific and general homology between the mammalian and chicken enzymes suggest that the gene cloned is a chicken sEH homolog. A six histidine tag was encoded on the 3′ end of the construct for purification purposes. Recombinant enzyme was then produced in order to see if the transcript encoded for a protein with epoxide hydrolase activity.

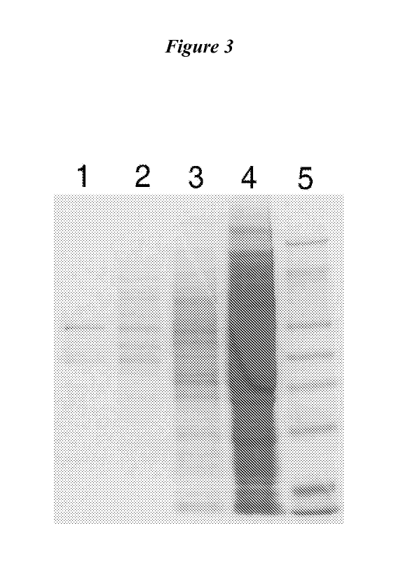

The tagged construct was expressed in a baculovirus expression system and purified on a nickel chelation column to a maximum purity of 75 percent (FIG. 3). Radioactively labeled t-DPPO was chosen as the initial substrate to test for epoxide hydrolase activity (Borhan, B. et al., Anal. Biochem. 231:188-200 (1995)). The resulting purified recombinant enzyme was found to have a lower specific activity than either mouse or human sEH when assayed for EH activity using t-DPPO. The specific activity of the chicken enzyme was approximately twenty times lower than values previously reported for mouse and five times lower than values reported for human (Table 1). It was possible that the histidine tag added to the recombinant enzyme interfered with the EH activity. It was decided to test the effect of the tag on EH activity by examining the pattern of inhibition of EH activity in chicken liver crude extract compared to the purified recombinant enzyme.

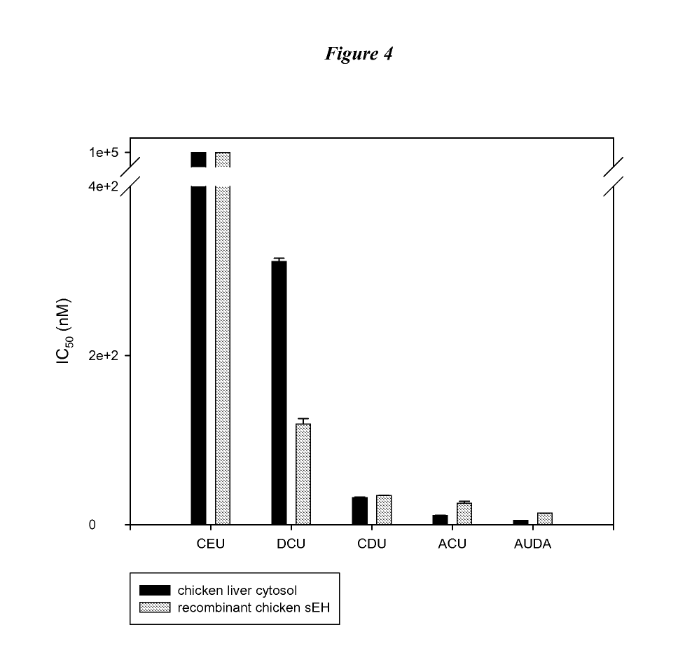

Six inhibitors were chosen that have been shown to have high, moderate and low IC₅₀s when tested against human enzyme (FIG. 4) (Morisseau, C. et al., Biochem. Pharmacol. 63:1599-1608 (2002)). The t-DPPO activity was used as a measure of EH activity for both the crude extract and purified recombinant enzyme was tested. It was found that the recombinant enzyme possessed the same relative response to inhibition as the chicken liver crude extract, giving evidence that the tag did not interfere with the EH activity. In some cases the IC₅₀ values obtained with the cytosol were different that the values obtained with the purified recombinant enzyme, for example, with DCU. This difference could be due to a number of factors. The purified protein solution lacks enzymes which might bind or degrade the inhibitor. This purified prep also lacks proteins or small molecules which might interact with sEH and modulate the catalytic activity of the enzyme. For these reasons, some difference in IC₅₀ values between the cytosol and purified recombinant enzyme can be expected.

Of the inhibitors tested with the recombinant enzyme, AUDA was the most potent. It possessed an IC₅₀ 13.7 nM when assayed with the recombinant enzyme. This indicates that this urea based inhibitor may be a good choice for in vivo inhibition of the chicken enzyme.

Since the histidine tag did not interfere with EH activity, the reduced activity of the chicken enzyme is probably due to structural differences between the mammalian and chicken sEH. The spacing between catalytic triad residues is highly conserved among mammalian enzymes (Beetham, J. K. et al., DNA Cell Biol. 14:61-71 (1995)). The distance between the catalytic aspartate and the orienting acid is 160-161 residues in rat, mouse and human sEH. This distance in the chicken sEH is 165 residues. It is possible that this difference in spacing is responsible for the attenuated epoxide hydrolase activity in the chicken enzyme.

To determine if the identified sEH homologue was responsible for the majority of the epoxide hydrolase activity detected in chicken crude extract, purified recombinant enzyme and chicken liver crude extract were run side by side on an IEF gel. Each lane was cut into 0.5 and 0.2 cm bands and assayed for t-DPPO activity. All of the recovered activities in both the purified recombinant and liver crude extract lanes were located in single 0.2 cm bands corresponding to the pI range of 6.0 to 6.2.

The purified recombinant enzyme has an experimentally determined molecular weight of 63 kDa and a PI of 6.1. When assayed with t-DPPO, the enzyme displays maximal epoxide hydrolase activity around pH 7.4. The half-life of epoxide hydrolase activity is over 6 d when the enzyme is kept at 4° C. The half-life at 25° C. is between 9 and 24 h, while the half-life at 37° C. is under 3 h.

The k_{cat} and Vₘₐₓ of the enzyme for t-DPPO was then determined. The chicken enzyme has a higher Km and lower k_{cat} for t-DPPO than either the recombinant mouse or human sEH (Table 1). Examining the k_{cat} to Vₘₐₓ ratio, it was found that t-DPPO was not a good substrate for the chicken enzyme compared to the mouse or human enzyme, having a value of 100 and 20 fold lower when compared to the mouse or human enzymes, respectively (Table 1). t-DPPO is not an endogenous substrate for the mammalian sEH. It does not possess the long alkyl chain present in proposed endogenous fatty acid substrates of the mammalian enzymes such as the EETs (Table 1). For this reason, the chicken enzyme was tested for epoxide hydrolase activity using a number of the EETs, as well as the fatty acid epoxide trans-9,10-epoxystearate.

The avian enzyme did not hydrolyze any EET or the epoxystearate substrate as readily as it did t-DPPO (Tables 1, 2). The enzyme showed the least activity towards cis-9,10-epoxystearate. The enzyme hydrolyzed the EETs at higher rates, with 14,15-EET being the best substrate (Table 2), albeit the activity was less than 1/30th the activity measured with t-DPPO. The enzyme hydrolyzed 11,12- and 8,9-EET at nearly equal rates (Table 2). Both the mouse and chicken enzymes hydrolyze 14,15-EET at over twice the rate of 11,12-, and 8,9-EET (Table 2).

Although residues thought to be important to the mammalian sEH phosphatase activity were not conserved in the chicken sEH, this activity has a potential role in the regulation of blood pressure (Arand, M. et al., Drug Metab. Rev. 35:365-383 (2003)). For this reason, the phosphatase activity of the chicken enzyme was assayed using the substrate threo-9,10-phosphonooxy-hydroxy-octadecanoic acid. The chicken enzyme did not hydrolyze this substrate under the conditions developed for the mammalian enzyme phosphatase activity assay.

The EETs are important endothelial derived vasoactive signaling molecules in mammals. The mammalian sEH has been shown to convert the EETs to their corresponding diols, the DHETs. Through this epoxide hydrolase activity, the mammalian sEH plays a role in blood pressure regulation. In this study, a chicken sEH homologue was identified and the epoxide hydrolase activity of the recombinant enzyme was assayed using the EETs and other substrates of the mammalian enzyme. It was found that the chicken enzyme had similar activities to the mammalian enzymes. It was also found that the recombinant enzyme was inhibited by a number of urea based inhibitors. AUDA was the most potent of the inhibitors in this series of experiments, and could be used to inhibit the enzyme in vivo. This would be a valuable tool to probe the role of sEH in endothelial derived vasodilation in chicken. In particular, a sEH inhibitor could be used in chicken models of PH, where it is believed that endothelial derived vasodilation has been impaired.

TABLE 1
Kinetic parameters using t-DPPO as a substrate

| | Kinetic | Recombinant | Recombinant | Recombinant |
|---|---|---|---|---|
| Structure of t-DPPO | parameter | chicken sEH | mouse sEH | human sEH |
| 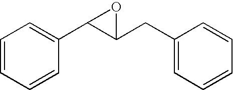 | Specific activity(nmol · min⁻¹ · mg⁻¹)Kₘ (μM)k_{cat} (s⁻¹)k_{cat}/Kₘ(μM⁻¹ · s⁻¹) | 823.1 ± 27.3 25.3 ± 0.9 0.9 ± 0.030.04 | 17000 ± 300.0 4.3 ± 0.618.0 ± 0.3 4.2 | 4500 ± 200.0 6.2 ± 0.64.3 ± 0.30.7 |

Recombinant chicken sEH was partially purified as described. Assay conditions are described in the Materials and Methods section. Results are presented as the mean ±standard deviation of 3 separate experiments. Values for the human and mouse enzyme are from (Morisseau, C. et al., Arch. Biochem. Biophys. 378:321-332 (2000))

TABLE 2
Specific activity of substrates

| | | Specific activity (nmol · min⁻¹ · mg⁻¹) | |
|---|---|---|---|
| | | Recombinant | Recombinant mouse* |
| Compound name | Structure | chicken sEH | and mouse sEH |
| cis-9,10-epoxystearic acid | 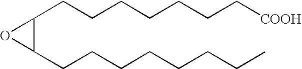 | 3.1 ± 0.2 | 1139 ± 34* |
| 14,15-EET | 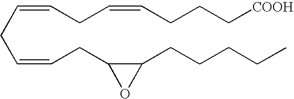 | 24.5 ± 2.1 | 1260.0 |
| 11,12-EET | 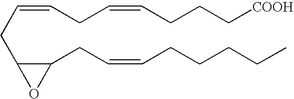 | 12.0 ± 1.2 | 640.0 |
| 8,9-EET | 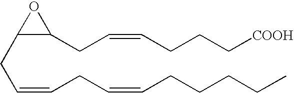 | 11.6 ± 0.1 | 370.0 |

Recombinant chicken sEH was partially purified as described. Assay conditions are described in the Materials and Methods section. Results are presented as the mean ±standard deviation of 3 separate experiments. Values for the mouse enzyme assayed with cis-9,10-epoxystearic are from (Morisseau, C. et al., Arch. Biochem. Biophys. 378:321-332 (2000)). Values for the mouse enzyme assayed with the EETs are from (Chacos, N. et al., Arch. Biochem. Biophys. 223:639-648 (1983))

### Example 2

The following example demonstrates identifying inhibitors of chicken sEH with low IC50 values.

### Materials and Methods

### Inhibitor Assays

Inhibitors were tested for their IC₅₀ values using partially purified recombinant chicken sEH (130 μL) at a concentration of 0.4 ng/μL. Dilutions of enzyme, inhibitors and substrate were made in BisTris-HCl buffer (25 mM, pH 7.0, 0.1 mg/ml BSA). The enzyme was incubated with 20 μL of inhibitor dilutions for 10 min prior to substrate addition. After this incubation, 50 μL of (3-phenyl-oxiranyl)-acetic acid cyano-(6-methoxy-naphthalen-2-yl)-methyl ester (PHOME) at a final concentration of 50 μM (1% final DMSO content per well) was added. Appearance of the reporter molecule 6-methoxy-2-naphthaldehyde was detected at room temperature with a SpectraMax M2 microplate reader (Molecular Devices, Sunnyvale, Calif.) and the following instrumental settings: excitation wavelength 316 nm, emission wavelength 460 nm (cutoff 455 nm), 3 reads per well.

The inhibitor screen occurred in two steps. The first step was incubation with a final inhibitor concentration of 100 nM. Positive hits obtained in this primary screen were then incubated with inhibitors at final concentrations of 1, 10, and 100 nM. For compounds selected in the secondary screen, IC50s were determined by linear regression analyses employing at least 3 data points at different concentrations in the linear range of the resulting inhibition curve (between 20 and 80% enzyme activity reduction) using final inhibitor concentrations from 0.0004 μM to 0.05 μM.

### Results

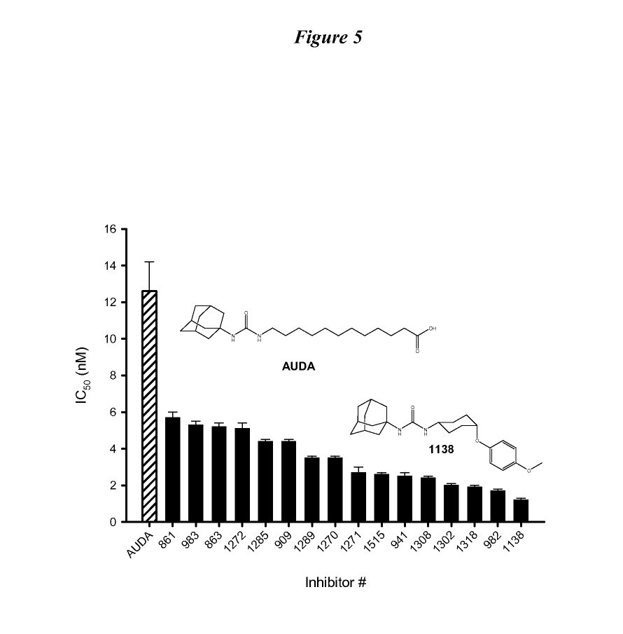

A library of 1320 compounds was screened. The library also included public domain compounds, for example, triclocarban. From this library, several compounds were selected as inhibitors of chicken sEH in vivo (Table 3 and FIG. 5).

TABLE 3
Inhibitory Compounds of Chicken sEH

| | Compound | Chicken sEH |
|---|---|---|
| Structure | number | IC₅₀ (nM) |
| 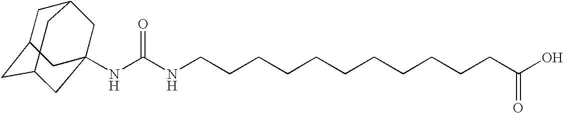 | 700 | 12.6 ± 1.6 |
| 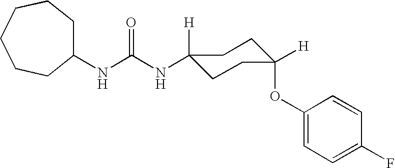 | 1515 | 2.6 ± 0.1 |
| 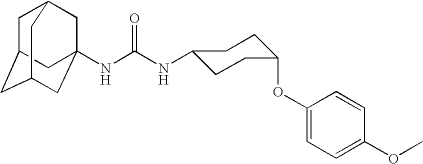 | 1138 | 1.2 ± 0.1 |
| 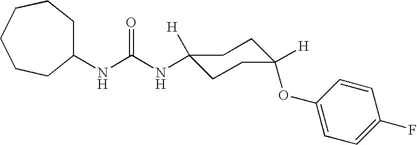 | 1271 | 2.7 ± 0.3 |
| 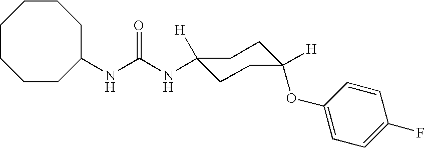 | 1272 | 5.1 ± 0.3 |
| 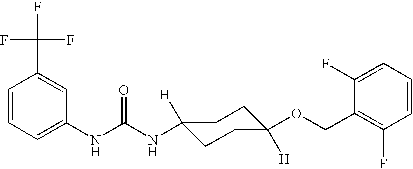 | 1285 | 4.4 ± 0.1 |
| 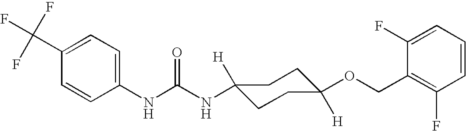 | 1289 | 3.5 ± 0.1 |
| 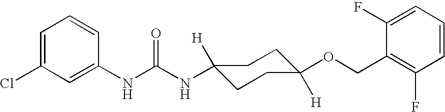 | 1302 | 2.0 ± 0.1 |
| 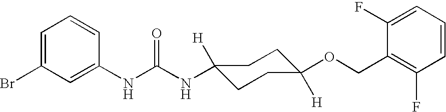 | 1308 | 2.4 ± 0.1 |
| 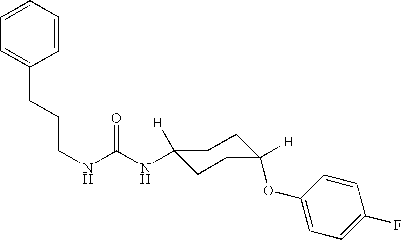 | 1270 | 3.5 ± 0.1 |
| 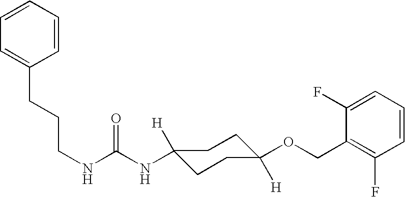 | 1318 | 1.9 ± 0.1 |
| 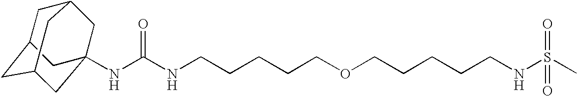 | 941 | 2.5 ± 0.2 |
| 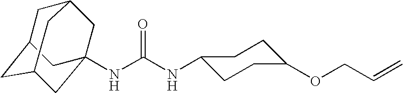 | 982 | 1.7 ± 0.1 |
| 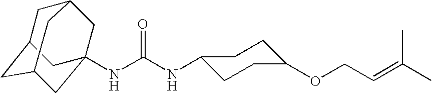 | 983 | 5.3 ± 0.2 |
| 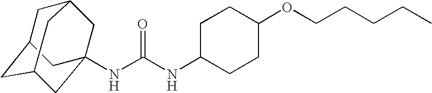 | 909 | 4.4 ± 0.1 |
| 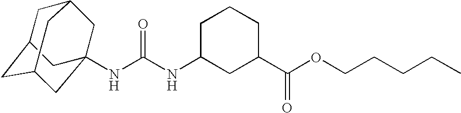 | 861 | 5.7 ± 0.3 |
| 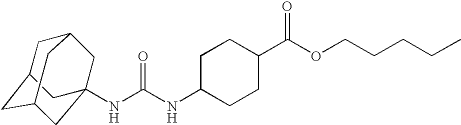 | 863 | 5.2 ± 0.2 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting or preventing pulmonary hypertension syndrome or a symptom thereof in an avian subject in need thereof, said method comprising administering to said avian subject an effective amount of an inhibitor of soluble epoxide hydrolase (“sEH”), thereby inhibiting pulmonary hypertension syndrome or said symptom thereof in said avian subject.

2. The method of claim 1 comprising further co-administering a cis-epoxyeicosantrienoic acid (“EET”).

3. The method of claim 1, wherein the avian subject is of the Subclass Neognathae.

4. The method of claim 1, wherein the avian subject is of the Order Galliformes.

5. The method of claim 1, wherein the avian subject is of the Family Phasianidae.

6. The method of claim 1, wherein the avian subject is a chicken (Gallus).

7. The method of claim 1, wherein the symptom thereof is pulmonary hypertension.

8. The method of claim 1, wherein the symptom thereof is right-sided congestive heart failure.

9. The method of claim 1, wherein the symptom thereof is hypoxemia.

10. An isolated nucleic acid encoding a soluble epoxide hydrolase having at least 95% nucleic acid sequence identity to the nucleic acid sequence depicted in FIG. 1.

11. An isolated soluble epoxide hydrolase having at least 95% amino acid sequence identity to the amino acid sequence depicted in FIG. 1.

12. An isolated soluble epoxide hydrolase of claim 11 comprising the amino acid sequence depicted in FIG. 1.
